(19) United States
(12) Patent Application Publication
(10) Pub. No.: US 2026/0169001 A1
(43) Pub. Date: Jun. 18, 2026

(54) METHOD OF MEASURING TARGET SUBSTANCE, AND REAGENT THEREFOR

(71) Applicant: CANON KABUSHIKI KAISHA, Tokyo (JP)

(72) Inventors: SHUHEI ISHIOKA, Kanagawa (JP); TSUTOMU SHIMANO, Shizuoka (JP); SAYAKA OONUKI, Ibaraki (JP); RISA WAKISAKA, Tokyo (JP); KIYOTAKE FUNAYAMA, Tokyo (JP); KENGO KANAZAKI, Kanagawa (JP)

(21) Appl. No.: 19/409,061

(22) Filed: Dec. 4, 2025

(30) Foreign Application Priority Data
Dec. 13, 2024 (JP) 2024-218590
Nov. 4, 2025 (JP) 2025-185679

(51) Int. Cl. G01N 33/68; G01N 21/31; G01N 21/77; G01N 33/543

(52) U.S. Cl. CPC G01N 33/68; G01N 21/31; G01N 21/77; G01N 33/54346

(57) ABSTRACT

Provided are a measurement method that improves a detection limit and enables highly reproducibility at a high concentration, and a reagent therefor. The method is for measuring a target substance, the target substance being a protein complex, the method including: mixing the specimen and a first reagent to provide a first solution; mixing the first solution and a second reagent, which includes a particle immobilized a ligand that binds to the target substance, to provide a second solution; and measuring an absorbance of the second solution, wherein the absorbance at a time point of 35 to 45 seconds from the second mixing step is 1.40 or more. The method is characterized in that the amount of change in absorbance in the low-concentration region of the target substance is large, and the amount of change in absorbance in the high-concentration region is suppressed to fall within a specified range.

### BACKGROUND

### Field of the Technology

The present disclosure relates to a method of measuring a target substance, and a reagent therefor.

### Description of the Related Art

There is given immunoturbidimetry using a particle as a simple and rapid immunological testing method. In this method, a dispersion of a particle to which a ligand having affinity to a target substance is bonded, and a specimen that may contain the target substance are mixed. At this time, the particle causes an aggregation reaction in accordance with the amount of the target substance in the specimen, and hence the target substance can be qualitatively or quantitatively determined with a device configured to optically detect the aggregation reaction as a variation in, for example, scattered light intensity, transmitted light intensity, or absorbance.

In an immunological measurement method by immunoturbidimetry using a particle, a device configured to measure the absorbance of a reaction liquid is used. It is known that the device has a maximum measurable value of the absorbance, and hence an absorbance equal to or higher than the maximum value cannot be measured (Japanese Patent Laid-Open No. 2004-191322 and Japanese Patent Laid-Open No. 2017-83440). Meanwhile, a specific absorbance range and conditions under which stable measurement is possible have not been clarified.

The immunoturbidimetry using a particle is a simple and very rapid evaluation method, but investigations for measuring a low-concentration target substance have not sufficiently progressed. As a method of measuring a low-concentration target substance, a chemiluminescent enzyme immunoassay is generally used.

The chemiluminescent enzyme immunoassay is a method in which an antigen is caused to react with an antibody immobilized on a solid phase, then an enzyme-labeled antibody is caused to secondarily react with the antigen, and a chemiluminescent substrate is added to measure the luminescence intensity of the enzyme-labeled antibody. The chemiluminescent enzyme immunoassay allows measurement of a low-concentration target substance, but the procedure thereof is more complicated than that of the immunoturbidimetry using a particle, and hence the chemiluminescent enzyme immunoassay is a method that requires measurement time and cost.

### SUMMARY

In the immunoturbidimetry using a particle, it has been demanded to improve detection sensitivity to a lower-concentration target substance.

The present disclosure relates to a measurement method for measuring a target substance in a specimen, the target substance being a protein forming a complex that is hexameric or higher, the measurement method including: a first mixing step of mixing the specimen and a first reagent to provide a first mixed solution; a second mixing step of mixing the first mixed solution and a second reagent, which includes a particle having immobilized thereon a ligand that specifically binds to the target substance, to provide a second mixed solution; and a measurement step of measuring an absorbance of the second mixed solution, wherein the absorbance at a time point of 35 seconds or more and 45 seconds or less from the mixing in the second mixing step is 1.40 or more, and wherein, when an amount of change in absorbance of a specimen A containing A ng/ml of the target substance is represented by ΔODA, an amount of change in absorbance of a specimen B containing B ng/ml of the target substance is represented by ΔODB (B>A), and a relationship of symbols is represented by (ΔODB−ΔODA)/(B−A)=X, an X of 0.00100 or more is present in a range where both A and B are 0 or more and 100 or less, and an X of 0.00003 or more and 0.00010 or less is present in a range where both A and B are 1,000 or more and 1,500 or less.

The present disclosure also relates to a reagent for measuring a target substance in a specimen, the target substance being a protein forming a complex that is hexameric or higher, the reagent including a first reagent including a buffering agent, and a second reagent which includes a particle having immobilized thereon a ligand that specifically binds to the target substance, wherein, when a first mixing step of obtaining a first mixed solution, in which the specimen and the first reagent are mixed at a volume ratio “specimen:first reagent” of 15:60, and a second mixing step of obtaining a second mixed solution, in which the first mixed solution and the second reagent are mixed at a volume ratio “first mixed solution:second reagent” of 75:30, are performed, an absorbance of the second mixed solution at a time point of 35 seconds or more and 45 seconds or less from the mixing in the second mixing step when using the specimen containing 0 ng/ml of the target substance is 1.40 or more, and wherein, when an amount of change in absorbance of the second mixed solution from 40 seconds to 300 seconds from the mixing in the second mixing step when using the specimen containing 0 ng/ml of the target substance is represented by ΔOD(0), an amount of change in absorbance of the second mixed solution from 40 seconds to 300 seconds from the mixing in the second mixing step when using the specimen containing 10 ng/ml of the target substance is represented by ΔOD(10), an amount of change in absorbance of the second mixed solution from 40 seconds to 300 seconds from the mixing in the second mixing step when using the specimen containing 90 ng/mL of the target substance is represented by ΔOD(90), an amount of change in absorbance of the second mixed solution from 40 seconds to 300 seconds from the mixing in the second mixing step when using the specimen containing 100 ng/ml of the target substance is represented by ΔOD(100), an amount of change in absorbance of the second mixed solution from 40 seconds to 300 seconds from the mixing in the second mixing step when using the specimen containing 1,000 ng/ml of the target substance is represented by ΔOD(1,000), an amount of change in absorbance of the second mixed solution from 40 seconds to 300 seconds from the mixing in the second mixing step when using the specimen containing 1,100 ng/ml of the target substance is represented by ΔOD(1,100), an amount of change in absorbance of the second mixed solution from 40 seconds to 300 seconds from the mixing in the second mixing step when using the specimen containing 1,400 ng/ml of the target substance is represented by ΔOD(1,400), and an amount of change in absorbance of the second mixed solution from 40 seconds to 300 seconds from the mixing in the second mixing step when using the specimen containing 1,500 ng/ml of the target substance is represented by ΔOD(1,500), a value of (ΔOD(10)−ΔOD(0))/(10−0) or (ΔOD(100)−ΔOD(90))/(100−90) is 0.00100 or more, and a value of (ΔOD(1,100)−ΔOD(1,000))/(1,100−1,000) or (ΔOD(1,500)−ΔOD(1,400))/(1,500−1,400) is 0.00003 or more and 0.00010 or less.

Features of the present disclosure will become apparent from the following description of embodiments. The following description of embodiments is described by way of example.

### DESCRIPTION OF THE EMBODIMENTS

Embodiments of the present disclosure are described in detail below. However, the technical scope of the present disclosure is not limited to the embodiments. A particle on which a ligand that specifically binds to a target substance is immobilized is hereinafter also referred to as “affinity particle,” and a particle before the ligand that specifically binds to the target substance is immobilized thereon is also referred to as “pre-sensitization particle.”

In response to the above-mentioned object, the inventors have made investigations to improve detection sensitivity to a low-concentration target substance in immunoturbidimetry using a particle. As a result, it has been found that, when a protein forming a complex that is hexameric or higher is used as the target substance, in particular, as a particle concentration in a reagent is increased, the amount of change in absorbance is increased and the detection sensitivity to the low-concentration target substance improves. In particular, it has been found that the effect is more excellent when the target substance is ferritin forming a complex that is hexameric or higher.

This is conceived to be caused by the number of ligand recognition sites of the target substance. In the case of a protein forming only a monomer or a complex that is pentameric or lower, the number of ligand recognition sites is also small. Accordingly, when the target substance and a reagent are mixed, the ligand recognition sites are easily consumed by reacting with the target substance. As a result, as the reaction progresses, the number of ligand recognition sites decreases, and thus the aggregation amount of the particles is unlikely to change even when the particle concentration in the reaction system is high. Meanwhile, in the case of a protein forming a complex that is hexameric or higher, the number of ligand recognition sites is large. For example, ferritin is a complex of 24 proteins and may have 24 antibody recognition sites. Even when the reaction progresses, the ligand recognition sites tend to remain, and when the particle concentration in the reaction system is high, multiparticle aggregation in which multiple particles react with one target substance is likely to occur. As a result, it is conceived that, as the particle concentration in the reagent is increased, the amount of change in absorbance is increased and the detection sensitivity to the low-concentration target substance improves.

However, it has been found that when the particle concentration is increased in the measurement of a protein forming a complex that is hexameric or higher, the measurement reproducibility in the high-concentration region is decreased. It has been conceived that this is caused by an increase in measurement error due to an excessive increase in measurement absorbance when a high-concentration target substance is measured. In particular, when the protein forming a complex that is hexameric or higher is used as the target substance, it is conceived that multiparticle aggregation mediated by a plurality of target substances and a plurality of particles occurs. It has been conceived that the number of particles involved in the aggregation is unstable, and in addition, the measurement absorbance is high, and hence the measurement error is amplified, leading to a further decrease in measurement reproducibility.

Based on the above-mentioned idea, reagent design has been performed so that the amount of change in absorbance in the high-concentration region is intentionally suppressed to fall within a certain range, and as a result, it has been found that measurement in the high-concentration region is also possible and highly reproducible measurement can be performed. Those are based on an object and a design idea that have first been found by thoroughly investigating the improvement of the detection sensitivity to a low-concentration target substance by immunoturbidimetry using a particle for the measurement of a protein forming a complex that is hexameric or higher, and it can be said that the design is unique and different from conventional ideas.

In the immunoturbidimetry using a particle, when a protein that is hexameric or higher is used as a target substance, the particle concentration is increased to a certain level or more to increase the amount of change in absorbance in the low-concentration region, and the amount of change in absorbance in the high concentration is set within a certain range, to thereby achieve highly reproducible measurement in which measurement is also possible in the high-concentration region. As a result, it is conceived that measurement can be performed with high measurement reproducibility in a wide range of concentration regions from the low-concentration region to the high-concentration region.

From the above-mentioned idea, the present disclosure is directed to a measurement method for measuring a target substance in a specimen, the target substance being a complex that is hexameric or higher, the measurement method including: a first mixing step of mixing the specimen and a first reagent to provide a first mixed solution; a second mixing step of mixing the first mixed solution and a second reagent, which includes a particle having immobilized thereon a ligand that specifically binds to the target substance, to provide a second mixed solution; and a measurement step of measuring an absorbance of the second mixed solution, wherein the absorbance at a time point of 35 seconds or more and 45 seconds or less from the mixing in the second mixing step is 1.40 or more, and wherein, when an amount of change in absorbance of a specimen A containing A ng/mL of the target substance is represented by ΔODA, an amount of change in absorbance of a specimen B containing B ng/ml of the target substance is represented by ΔODB (B>A), and a relationship of symbols is represented by (ΔODB−ΔODA)/(B−A)=X, an X of 0.00100 or more is present in a range where both A and B are 0 or more and 100 or less, and an X of 0.00003 or more and 0.00010 or less is present in a range where both A and B are 1,000 or more and 1,500 or less.

In this case, the amount of change in absorbance refers to an amount of change in absorbance of the second mixed solution from the mixing in the second mixing step to the measurement step.

In this embodiment, the mixing of the specimen and the first reagent may refer to the addition of one of the specimen or the first reagent to the other, or may refer to the addition of the specimen and the first reagent to each other. The same applies to any other mixing in the present specification. In addition, the phrase “from the mixing” in this embodiment means from the time of the completion of stirring when the entire amount of the two kinds of liquids is mixed and the stirring is performed to sufficiently mix the two kinds of liquids. In addition, a time period from the time of the start of the mixing of the two kinds of liquids to the time of the completion of the stirring described above is 5 seconds or less. The stirring may be performed with a stirring bar, by irradiation with an ultrasonic wave, or by tapping. For example, the sentence “the absorbance at a time point of 35 seconds or more and 45 seconds or less from the mixing is 1.40 or more” means that a case in which the absorbance measured at a time point of 35 seconds or more and 45 seconds or less from the time of the completion of the stirring is 1.40 or more is present. For the term “mixing”, the same applies to any other mixing in the present specification.

X is hereinafter also referred to as “magnitude of the amount of change in absorbance with respect to the concentration.”

The sentence “the absorbance of the second mixed solution at a time point of 35 seconds or more and 45 seconds or less from the mixing in the second mixing step is 1.40 or more” means that the particle concentration is high. At this time, excellent detection sensitivity is obtained when a protein that is hexameric or higher is used as the target substance. Meanwhile, when the measurement absorbance is low, it can be said that the particle concentration is low, and thus this case does not pertain to the object of the present disclosure. The measurement absorbance is not excessively increased even when the amount of change in absorbance is large through a sufficient reaction with a high-concentration specimen.

In addition, the presence of an X of 0.00100 or more in the range where both A and B are 0 or more and 100 or less means that the magnitude of the amount of change in absorbance with respect to the concentration is large in the low-concentration region. Accordingly, when the above-mentioned condition is satisfied, measurement can be performed with higher sensitivity in the low-concentration region.

Further, the presence of an X of 0.00003 or more in the range where both A and B are 1,000 or more and 1,500 or less means that the magnitude of the amount of change in absorbance with respect to the concentration is equal to or more than a certain level in the high-concentration region. In addition, the presence of an X of 0.00010 or less in the range where both A and B are 1,000 or more and 1,500 or less means that the amount of change in absorbance at a high concentration is suppressed, and the measurement absorbance is not excessively increased even after a sufficient reaction during measurement in the high-concentration region. Thus, the measurement error is reduced, and hence a result with excellent measurement reproducibility is obtained. In the present disclosure, ΔODA<ΔODB is always satisfied in the range where both A and B are 1,000 or more and 1,500 or less and in the range where the difference between A and B is 100 or more.

### <Detection Method and Measurement Method>

A measurement method to be used in the present disclosure is immunoturbidimetry using a particle. The immunoturbidimetry using a particle is a method of optically detecting inter-particle aggregation that occurs when the affinity particle of the present disclosure and the specimen are mixed. In the present disclosure, an absorbance is used as a method of detecting the optical change. There is no limitation on an instrument used for the measurement, and any optical instrument capable of detecting the absorbance may be used. Of those, a general-purpose automatic analyzer, which is simple to control the dispensing amount of the specimen or the reagent, a mixing time, a measurement wavelength, and the like, is preferably used.

An example of the measurement method using the automatic analyzer is described below. First, 8 μL to 25 μL of the specimen is dispensed into a reaction container. Next, as the first mixing step, 50 μL to 150 μL of the first reagent is dispensed into the same reaction container, and after stirring and mixing, the temperature is adjusted to a predetermined temperature, and the container is kept warm for from 180 seconds to 600 seconds. The temperature at this time preferably falls within the range of from 20° C. to 50° C.

After that, as the second mixing step, 10 μL to 150 μL of the second reagent is dispensed into the same reaction container, and after stirring and mixing, a reaction is performed for from 180 seconds to 600 seconds. At this time, it is preferred to start the second mixing step after 280 seconds or more have elapsed from the mixing in the first mixing step. When 280 seconds elapse from the mixing in the first mixing step, the specimen and the first reagent are uniformly mixed, and hence the particle aggregation becomes uniform and the measurement reproducibility improves. In addition, the reaction time of the second mixing step is more preferably 200 seconds or more and 600 seconds or less.

Further, the absorbance of the second mixed solution is measured, and the amount of change in absorbance is calculated. At this time, the measurement wavelength of the absorbance is preferably 500 nm or more and 750 nm or less. In addition, the measurement wavelength may be combined with another wavelength. When the wavelength in this range is included, the amount of change in absorbance tends to be large even for a low-concentration target substance, and the low-concentration sensitivity tends to improve. Further, the measurement reproducibility is excellent because the measurement accuracy of the absorbance is high. In this embodiment, the amount of change in absorbance ΔOD may be calculated from the difference between the absorbance within a range of 15 seconds or more and 45 seconds or less from the mixing in the second mixing step, and the absorbance within a range of 250 seconds or more and 300 seconds or less from the mixing in the second mixing step.

The concentration of the affinity particle in the second mixed solution is preferably 0.02 mass/vol % or more and 0.10 mass/vol % or less. When the concentration is 0.02 mass/vol % or more, the amount of change in absorbance tends to be large at a low concentration for a protein that is hexameric or higher. In addition, when the concentration is 0.10 mass/vol % or less, the number of particles not involved in the aggregation is reduced, and the measurement absorbance becomes low. An error is unlikely to occur in absorbance measurement, and thus the measurement reproducibility is excellent.

The concentration of the specimen in the second mixed solution is preferably 7.0 vol % or more and 30.0 vol % or less. When the concentration of the specimen in the second mixed solution is less than 7.0 vol %, sufficient detection sensitivity cannot be obtained. Meanwhile, when the concentration is more than 30.0 vol %, the aggregation of the particles is affected by components other than the target substance in the specimen, and thus the measurement reproducibility becomes poor.

### <Specimen and Target Substance>

The specimen that may be used in the present disclosure is not particularly limited as long as the specimen contains the target substance, and examples thereof include blood, serum, and plasma.

The target substance of the present disclosure is a protein forming a complex that is hexameric or higher. In addition, the target substance is preferably a protein having a molecular weight of 300 kDa or more and 600 kDa or less. When the molecular weight is 300 kDa or more, the protein easily reacts with the antibody on a surface of the particle, and the aggregation reaction is stabilized. In addition, when the molecular weight is 600 kDa or less, the number of antigens per unit concentration increases, the number of aggregated particles increases, and the amount of change in measurement absorbance is stabilized. Accordingly, the effect of the present disclosure is easily obtained. Ferritin, which is given as an example in the present disclosure, forms a complex that is hexameric or higher and has a molecular weight of 445 kDa, and is therefore more preferred. The target substance of the present disclosure is preferably a substance having a plurality of reaction sites for an antibody (ligand) that specifically binds to the target substance, and ferritin is also preferable from this viewpoint.

### <First Reagent and Second Reagent>

An immunoturbidimetry reagent using a particle used in the measurement method of the present disclosure includes a first reagent including a buffering agent, and a second reagent including an affinity particle having immobilized thereon a ligand that specifically binds to the target substance. The first reagent is used for the role of diluting the specimen or subjecting the specimen to prevent non-specific reaction. Accordingly, the first reagent may include a buffering agent, a sugar, a surfactant, a sensitizer, or a non-specific reaction inhibitor as described later.

In this embodiment, when a first mixing step of obtaining a first mixed solution, in which the specimen and the first reagent are mixed at a volume ratio “specimen:first reagent” of 15:60, and a second mixing step of obtaining a second mixed solution, in which the first mixed solution and the second reagent are mixed at a volume ratio “first mixed solution:second reagent” of 75:30 are performed, the absorbance of the second mixed solution at a time point of 35 seconds or more and 45 seconds or less from the mixing in the second mixing step when using the specimen containing 0 ng/ml of the target substance is 1.40 or more, and when an amount of change in absorbance of the second mixed solution from 40 seconds to 300 seconds from the mixing in the second mixing step when using the specimen containing 0 ng/ml of the target substance is represented by ΔOD(0), an amount of change in absorbance of the second mixed solution from 40 seconds to 300 seconds from the mixing in the second mixing step when using the specimen containing 10 ng/ml of the target substance is represented by ΔOD(10), an amount of change in absorbance of the second mixed solution from 40 seconds to 300 seconds from the mixing in the second mixing step when using the specimen containing 90 ng/ml of the target substance is represented by ΔOD(90), an amount of change in absorbance of the second mixed solution from 40 seconds to 300 seconds from the mixing in the second mixing step when using the specimen containing 100 ng/ml of the target substance is represented by ΔOD(100), an amount of change in absorbance of the second mixed solution from 40 seconds to 300 seconds from the mixing in the second mixing step when using the specimen containing 1,000 ng/ml of the target substance is represented by ΔOD(1,000), an amount of change in absorbance of the second mixed solution from 40 seconds to 300 seconds from the mixing in the second mixing step when using the specimen containing 1,100 ng/ml of the target substance is represented by ΔOD(1,100), an amount of change in absorbance of the second mixed solution from 40 seconds to 300 seconds from the mixing in the second mixing step when using the specimen containing 1,400 ng/mL of the target substance is represented by ΔOD(1,400), and an amount of change in absorbance of the second mixed solution from 40 seconds to 300 seconds from the mixing in the second mixing step when using the specimen containing 1,500 ng/ml of the target substance is represented by ΔOD(1,500), a value of (ΔOD(10)−ΔOD(0))/(10−0) or (ΔOD(100)−ΔOD(90))/(100−90) is 0.00100 or more, and a value of (ΔOD(1,100)−ΔOD(1,000))/(1,100−1,000) or (ΔOD(1,500)−ΔOD(1,400))/(1,500−1,400) is 0.00003 or more and 0.00010 or less.

The pH of each of the first reagent and the second reagent is preferably 5.0 or more and 11.0 or less. When the pH falls within the above-mentioned range, the dispersed state of the affinity particle and the mixed state when the specimen is mixed can be uniformized. More excellent measurement accuracy is obtained. The pHs of the first reagent and the second reagent may be values different from each other.

It is preferred that the first reagent and the second reagent each include a buffering agent (buffer). The kind of the buffer is not particularly limited, and may be a substance from which a buffering capacity is obtained. For example, acetate, citrate, phosphate, Tris, glycine, borate, and carbonate buffers, and Good's buffers, such as MES, Bis-Tris, ADA, PIPES, ACES, MOPSO, BES, MOPS, TES, HEPES, TAPSO, POPSO, HEPSO, EPPS, Tricine, Bicine, TAPS, CHES, and CAPS, are suitably used. The buffering agents may be used alone or in combination thereof. In addition, the buffering agents used for the first reagent and the second reagent may be identical to or different from each other.

It is preferred that the first reagent and the second reagent each further include a sugar or a sugar alcohol. When the first reagent and the second reagent each include a sugar, the hydration of the surface of the affinity particle or the specimen component is promoted, an interaction between the affinity particle and the specimen component is reduced, and the measurement reproducibility improves. Examples of such sugar and sugar alcohol include, but not limited to: monosaccharides, such as glucose and fructose; disaccharides, such as sucrose, lactose, maltose, cellobiose, and trehalose; or oligosaccharides, such as maltotriose and dextran; and sugar alcohols, such as erythritol, mannitol, sorbitol, and xylitol. The sugars and the sugar alcohols may each be used alone or in combination thereof.

It is preferred that the first reagent and the second reagent each further include a surfactant. As the surfactant, a known nonionic surfactant, anionic surfactant, cationic surfactant, or amphoteric surfactant may be used. It is preferred that the first reagent and the second reagent each include one or more of a sorbitan fatty acid ester, a polyoxyethylene alkyl ether, and a polyoxyethylene phenyl ether out of those surfactants. Those surfactants each have high hydrophilicity and high solubilizing and stabilizing effects on the specimen component. Accordingly, the interaction between the affinity particle and the specimen component is reduced, and excellent measurement reproducibility is obtained. The concentration of the surfactant is preferably 0.001 mass % or more and 0.200 mass % or less in the second mixed solution.

The first reagent may further include a sensitizer. Examples of the sensitizer include water-soluble polymers, such as polyethylene glycol, carboxymethyl cellulose, methyl cellulose, polyvinylpyrrolidone, polyvinyl alcohol, polyacrylic acid, and polyglycosyl ethyl methacrylate.

In addition, the first reagent and the second reagent of the present disclosure may each include a chelating agent, such as EDTA, CyDTA, DTPA, EGTA, NTA, or NTP, a protein, such as bovine serum albumin, casein, or gelatin, a non-specific reaction inhibitor such as a protein degradation product, an amino acid, animal serum, an antibody, an antibody fragment, or a reducing agent, a stabilizer, such as a protein or a preservative, or an inorganic salt, such as sodium chloride, potassium chloride, or calcium chloride.

### <Affinity Particle and Pre-Sensitization Particle>

The volume average particle diameter of the affinity particle of the present disclosure is preferably 200 nm or more and 500 nm or less. When the volume average particle diameter is 200 nm or more, the number of aggregated particles required per unit amount of change in absorbance is reduced, and the action of the specimen component on the affinity particle becomes more uniform. In addition, when the volume average particle diameter is 500 nm or less, the amount of change in absorbance generated by the aggregation per particle is reduced, and thus a variation in the amount of change in absorbance due to a variation in the aggregation is unlikely to occur. Accordingly, excellent measurement reproducibility is obtained.

Further, it is preferred that the second reagent of the present disclosure include two kinds of affinity particles having different volume average particle diameters, and a difference in volume average particle diameter between the two kinds of affinity particles be 250 nm or less. When the second reagent includes two kinds of affinity particles having different volume average particle diameters, a wider range of target substance concentrations can be measured. At this time, when the difference in particle diameter is 250 nm or less, the reaction between the target substance and the affinity particle is unlikely to be biased, and even when a slight bias occurs, a difference in amount of change in absorbance is unlikely to occur, and thus excellent measurement reproducibility is obtained in a wide range of target substance concentrations.

In the present disclosure, when two kinds of affinity particles are used, the larger one is referred to as “large affinity particle” and the smaller one is referred to as “small affinity particle.” The volume average particle diameter of the large affinity particle is preferably 350 nm or more and 500 nm or less. When the diameter of the large affinity particle is 350 nm or more, the amount of change in absorbance due to the aggregation of the large affinity particle is large when the target substance has a low concentration, and the low-concentration sensitivity improves easily. When the diameter of the large affinity particle is 500 nm or less, the amount of change in absorbance per particle is small, and thus the variation in the amount of change in absorbance that occurs when the degree of aggregation varies is reduced, and excellent measurement reproducibility is obtained.

Further, the volume average particle diameter of the small affinity particle is preferably 200 nm or more and 300 nm or less. When the diameter of the small affinity particle is 200 nm or more, the size is such that the small affinity particle easily reacts with respect to the size of the target substance, and thus the reaction is stabilized, the variation in the amount of change in absorbance is reduced, and excellent measurement reproducibility is obtained. When the diameter of the small affinity particle is 300 nm or less, a plurality of target substances are unlikely to react with one particle when the target substance has a high concentration, and thus the amount of change in absorbance is stabilized, and excellent measurement reproducibility is obtained.

The zeta potential of the small affinity particle of the present disclosure is preferably −40.0 mV or more and −25.0 mV or less, and the zeta potential of the large affinity particle is preferably −35.0 mV or more and −15.0 mV or less. Further, it is preferred that the zeta potential of the small affinity particle be smaller than the zeta potential of the large affinity particle. When the zeta potential falls within the above-mentioned ranges, the balance between the electrostatic repulsion force and electrostatic attraction force of the affinity particle is appropriate. Accordingly, the dispersion of the affinity particle is stabilized, and excellent measurement reproducibility is obtained when the target substance has a high concentration.

The particle concentration ratio of the large affinity particle to the small affinity particle in the second reagent of the present disclosure is preferably 0.5 times or more and 2.0 times or less. When the particle concentration ratio falls within the above-mentioned range, a large difference does not occur in the particle concentration between the large affinity particle and the small affinity particle, and thus the possibility that the target substance is biased to react with any one of the particles is reduced, the reaction is stabilized, and the measurement reproducibility is excellent.

In the mixed system of the two kinds of affinity particles of the present disclosure, the ratio of the refractive index of the small affinity particle to the refractive index of the large affinity particle is preferably 0.87 or more and 0.97 or less. When there is a difference in refractive index between the two kinds of affinity particles within the above-mentioned range, highly sensitive measurement is possible from a lower concentration to a high concentration.

As the pre-sensitization particle used in the present disclosure, a conventionally known particle may be used. For example, polystyrene, a styrene-butadiene copolymer, a styrene-styrenesulfonate copolymer, or a styrene-glycidyl methacrylate copolymer may be used. Of those, a particle containing a polymer having a repeating unit represented by the following formula (1) is preferred as the pre-sensitization particle:

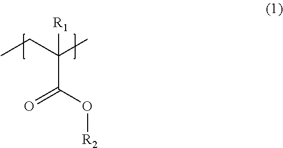

where R₁ represents a methyl group or a hydrogen atom, and R₂ represents a group having any one of an epoxy group, a hydroxy group, or a carboxy group.

When the pre-sensitization particle contains a polymer having the repeating unit represented by the formula (1), the surface of the affinity particle is easily hydrated, and the ionicity is low, and hence the influence of a hydrophobic ion component is suppressed. As a result, higher measurement accuracy is obtained. The formula (1) represents more preferably a structure represented by the following formula (1-A):

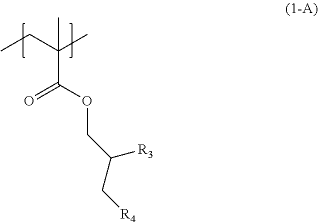

where at least one of R₃ or R₄ represents a hydroxy group, and the other thereof represents a hydroxy group or a group represented by the following formula (1-B):

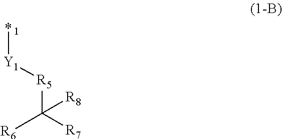

where R₅ represents a single bond or a methylene group, R₆, R₇, and R₈ are each selected from a hydrogen atom, a methyl group, a hydroxy group, a carboxy group, a hydroxymethyl group, and a carboxymethyl group, and at least one of R₆, R₇, or R₈ includes a hydroxy group or a carboxy group, Y₁ represents a sulfur atom or an imino group, and *₁ represents a bonding position with the structure represented by the formula (1-A).

Specific examples of the structure represented by the formula (1-A) are shown in the following formulae (1-A-1) to (1-A-12), but the present disclosure is not limited thereto.

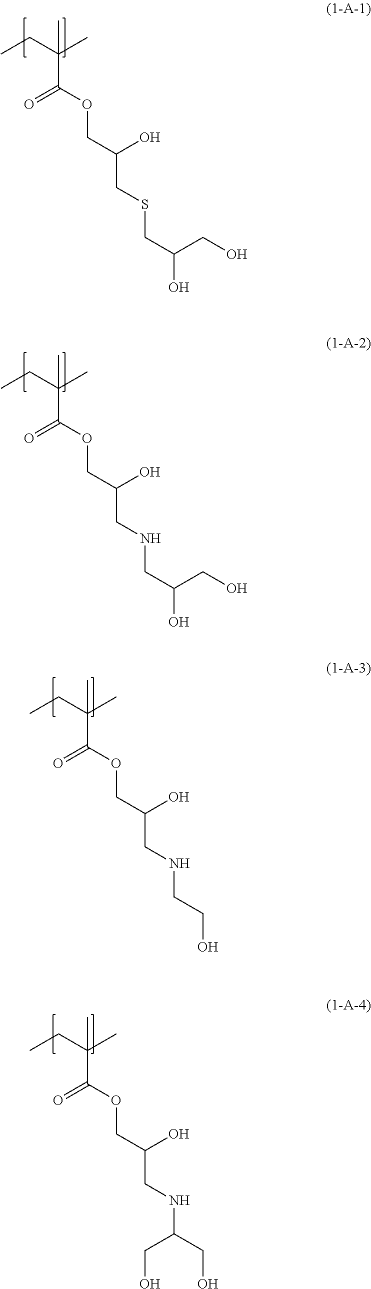

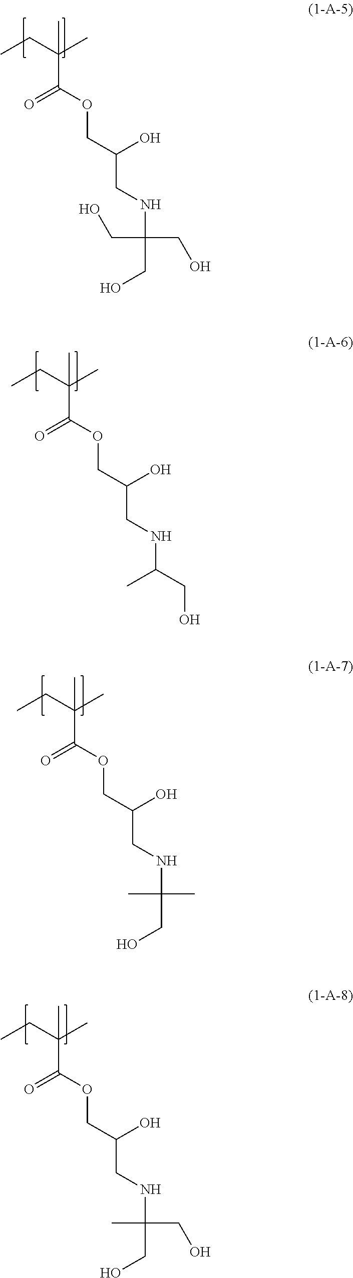

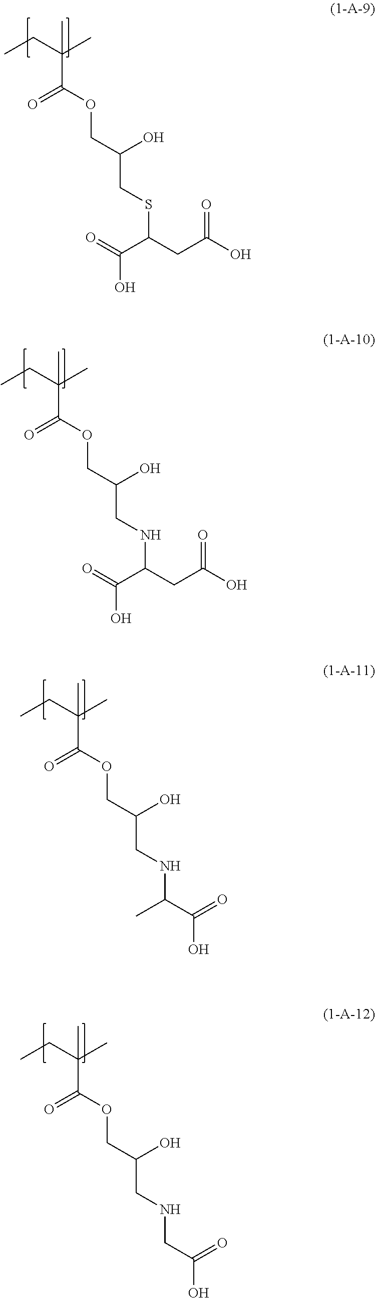

<Ligand that Specifically Binds to Protein>

The ligand is a compound that specifically binds to a receptor of a specific target substance. A site where the ligand binds to the target substance is fixed, and has selectively or specifically high affinity. Examples thereof include an antigen and an antibody, an enzyme protein and a substrate therefor, a signal substance, such as a hormone or a neurotransmitter, and a receptor therefor, a nucleic acid, and avidin and biotin, but the present disclosure is not limited thereto to the extent that the object of the present disclosure can be achieved. Specific examples of the ligand include an antigen, an antibody, an antigen-binding fragment (e.g., Fab, F(ab′)₂, F(ab′), Fv, or scFv), a naturally derived nucleic acid, an artificial nucleic acid, an aptamer, a peptide aptamer, an oligopeptide, an enzyme, and a coenzyme.

In this embodiment, the ligand is preferably an antibody or an antigen. Of those, an antibody having an isoelectric point of 5.0 or more and 8.0 or less is more preferred. The affinity particle using an antibody having an isoelectric point that falls within the range has a small charge on the surface of the affinity particle because of the influence of the antibody. As a result, an interaction between the affinity particle and the cationic component in the specimen is reduced, and excellent measurement accuracy is obtained. The isoelectric point may be measured by isoelectric focusing.

In the present disclosure, a method of immobilizing the ligand on the pre-sensitization particle may be performed by any known method, and the ligand can be immobilized by physically or chemically bonding the ligand to the pre-sensitization particle. For example, the ligand can be immobilized on the pre-sensitization particle by a covalent bond, a hydrogen bond, an ionic bond, electrostatic attraction, or a Van der Waals force. Examples of a method for the chemical bonding include a method involving utilizing a carbodiimide-mediated reaction or an NHS ester activation reaction, and a method of bonding a biotin-modified ligand to an avidin-bonded carboxy group.

The amount of the ligand with respect to 1.0 mg of the pre-sensitization particle is preferably 1.0 μg or more and 150.0 μg or less, more preferably 2.0 μg or more and 100.0 μg or less. Further, the affinity particle in the present disclosure is an affinity particle obtained by mixing two kinds of affinity particles, and it is still more preferred that the amounts of the ligand in the two kinds of affinity particles be different from each other.

In addition, the amount of the ligand with respect to 1.0 mg of the pre-sensitization particle in the second reagent is preferably 10.0 μg/mg or more and 40.0 μg/mg or less. When the amount is 10.0 μg/mg or more, the aggregation reaction between the target substance and the particle is stabilized, and when the amount is 40.0 μg/mg or less, multiparticle aggregation mediated by a plurality of target substances and a plurality of particles in the high-concentration region can be limited, and thus the measurement reproducibility is excellent.

Further, it is preferred that the amount of the ligand with respect to 1.0 mg of the pre-sensitization particle of the large affinity particle be 4.0 times or more and 20.0 times or less as large as the amount of the ligand with respect to 1.0 mg of the pre-sensitization particle of the small affinity particle. When the ratio of the amount of the ligand with respect to 1.0 mg of the pre-sensitization particle is 4.0 times or more, the reactivity of the large affinity particle to the target substance is higher than that of the small affinity particle. Accordingly, when mixed with a low-concentration target substance, the large affinity particle reacts predominantly, and the amount of change in absorbance becomes large. When the ratio of the amount of the ligand with respect to 1.0 mg of the pre-sensitization particle is 20.0 times or less, the reactivity of the small affinity particle to the target substance can be maintained. From those facts, when the ratio of the amount of the ligand with respect to 1.0 mg of the pre-sensitization particle falls within the above-mentioned ranges, the detection sensitivity and the measurement reproducibility to the low-concentration and high-concentration target substances are excellent.

An example of methods of measuring physical properties in the present disclosure is described below.

### <Method of Measuring Zeta Potential of Particle>

The zeta potential of the particle is measured with Zetasizer Nano ZS (Malvern Panalytical). The zeta potential in the present disclosure is measured under a state in which the particle is dispersed in a 0.01 N potassium hydroxide aqueous solution having a pH of 7.8 so that the concentration of the particle becomes 0.003 mass/vol %. The measurement conditions are as follows: 25° C., latex (n≈1.59) is selected as the refractive index of the particle, and pure water is selected as a solvent. The measurement is performed ten times, and the average value of the ten measured values is adopted as the zeta potential.

### <Method of Measuring Volume Average Particle Diameter of Particle>

The volume average particle diameter of the particle is measured with Zetasizer Nano ZS (Malvern Panalytical). The volume average particle diameter in the present disclosure is measured under a state in which the particle is dispersed in ion-exchanged water so that the concentration of the particle becomes 0.003 mass/vol %. The measurement conditions are as follows: 25° C., latex (n≈1.59) is selected as the refractive index of the particle, and pure water is selected as a solvent. The measurement is performed ten times, and the average value of the ten measured values is adopted as the volume average particle diameter.

In addition, a difference in volume average particle diameter when two or more kinds of particles having different particle diameters are mixed may be determined by observation with a scanning electron microscope or the like. Specifically, 300 or more particles are photographed at a magnification of 50,000 times, and the particle diameters of the 300 particles are measured by a known image processing method. The volume of each particle is determined from the measured particle diameter, and a volume distribution is prepared. A difference in particle diameter between the two kinds of particles may be determined by calculating the difference using the particle diameter that forms a peak in the prepared volume distribution as the volume average particle diameter of each particle.

<Method of Measuring Amount of Antibody with Respect to Particle (Antibody Sensitization Amount of Affinity Particle)>

A method of measuring the antibody sensitization amount of the affinity particle in the present disclosure is described. The antibody sensitization amount of the affinity particle with respect to the particle was determined by protein quantification. Herein, the term “antibody sensitization amount with respect to the particle” means the amount of the bound or adsorbed antibody per 1 mg of the particle.

First, 7 mL of the liquid A of Protein Assay BCA Kit (FUJIFILM Wako Pure Chemical Corporation) and 140 μL of the liquid B thereof were mixed, and the prepared liquid was adopted as a liquid AB. Next, 25 μL (particle concentration: 0.1 mass/vol %) of a dispersion of the affinity particle was added to 200 μL of the liquid AB, and the mixture was incubated at 60° C. for 30 minutes. After that, the resultant solution was centrifuged at 4° C. and 20,400×g for 5 minutes, and 200 μL of the supernatant was loaded into a 96-well microwell with a pipette. A standard sample, in which an antibody was mixed in a 10 mM HEPES buffer at an arbitrary concentration (five points within a concentration range of from 0 μg/mL to 200 μg/mL), was also loaded into a different location in the microwell in an amount of 200 μL, and the absorbance of the supernatant at 562 nm was collectively measured with a microplate reader, and the antibody amount was calculated from the calibration curve of the standard sample. The antibody amount (μg/mg) with respect to the particle was determined by dividing the calculated antibody amount by the mass of the particle.

### <Method of Measuring Refractive Index of Particle>

The refractive index of the particle is measured with Abbemat (manufactured by Anton Paar). The refractive index in the present disclosure is measured under a state in which the particle dispersion was dispersed so that its concentration became 5 mass/vol %. As the measurement conditions, a temperature of 25° C. and a measurement wavelength of 589.3 nm are selected. The refractive index of the particle is calculated from the Lorentz-Lorenz equation using the measured refractive index value, the specific gravity of the dispersion medium, the refractive index, and the specific gravity of the particle. The amount of ligand may also be determined by the same process.

### Examples

The present disclosure is described in detail below by way of Examples, but the present disclosure is not limited to these Examples.

### (Synthesis of Particle 1)

The synthesis of a particle 1 includes the following steps 1 to 3.

### (Step 1)

85.89 Grams of styrene (St: Kishida Chemical Co., Ltd.), 1.56 g of divinylbenzene (DVB: Kishida Chemical Co., Ltd.), and 1,190.67 g of ion-exchanged water were weighed into a 2 L four-necked separable flask to prepare a mixed solution. Oxygen was removed from the inside of the four-necked separable flask by holding the mixed solution at 70° C. under stirring at 200 rpm and performing a nitrogen flow at a flow rate of 200 mL/min. Next, a dissolved solution of 3.72 g of V-50 (FUJIFILM Wako Pure Chemical Corporation) dissolved in 50 g of ion-exchanged water, which had been separately prepared, was added to the mixed solution, and a polymerization reaction was performed for 48 hours to provide a dispersion containing a copolymer particle of St and DVB.

### (Step 2)

Next, 148.29 g of the dispersion diluted with ion-exchanged water so as to have a solid content concentration of 2.0 mass % was weighed into another four-necked separable flask. Next, 0.39 g of glycidyl methacrylate (GMA: Kishida Chemical Co., Ltd.) was added thereto. Oxygen was removed from the inside of the four-necked separable flask by holding the mixed solution at 70° C. under stirring at 100 rpm and performing a nitrogen flow at a flow rate of 200 mL/min. Then, a dissolved solution of 0.018 g of V-50 dissolved in 1 g of ion-exchanged water, which had been separately prepared, was added to the mixed solution. A dispersion containing a St/DVB/GMA composite particle was obtained by continuing stirring for 17 hours.

### (Step 3)

Finally, an aqueous solution having dissolved therein mercaptosuccinic acid (MSA: FUJIFILM Wako Pure Chemical Corporation) and 3-mercapto-1,2-propanediol (MPD: FUJIFILM Wako Pure Chemical Corporation), which had been prepared in advance, was added to the dispersion containing the St/DVB/GMA composite particle. At this time, the aqueous solution was prepared so that the molar ratio of 3-mercapto-1,2-propanediol to mercaptosuccinic acid was 6:4 (molar fraction), and the total number of moles of MSA and MPD was equal to the number of moles of glycidyl methacrylate. Subsequently, triethylamine (Kishida Chemical Co., Ltd.) was added thereto to adjust the pH to 10. Next, the dispersion was heated to 70° C. under stirring at 200 rpm, and the state was maintained for an additional 18 hours. Thus, a dispersion of a particle 1 was obtained. After that, the operation of separating the particle 1 from the dispersion with a centrifuge and re-dispersing the particle 1 in ion-exchanged water was repeated eight times to purify the particle 1, and finally, a particle 1 dispersion having a solid content concentration of 5.0 mass % was obtained. The volume average particle diameter of the obtained particle 1 was 420 nm.

### (Synthesis of Particle 2 to Particle 9, and Commercial Particles)

A particle 2 to a particle 9 were each synthesized by the same experimental operation as that in the method of synthesizing the particle 1 except that, in the method of synthesizing the particle 1, the amount of styrene, the amount of DVB, the amount of V-50, and the stirring speed used in the step 1, and the amount of GMA in the step 2 were changed as shown in Table 1. Those particles each have the structure represented by the formula (1) derived from GMA on a surface of the particle. In addition, a polystyrene particle IMMUTEX P0307 (JSR Life Sciences, LLC, carboxy-modified type) was prepared as a particle 10, and a polystyrene particle IMMUTEX 0113 (JSR Life Sciences, LLC, carboxy-modified type) was prepared as a particle 11. The physical properties of the obtained particle 1 to particle 9, and the particle 10 and the particle 11 are collectively shown in Table 1.

In each of the particles 1 to 9, the core particle included a styrene-divinylbenzene copolymer, and contained a polymer having the structural unit represented by the formula (1) on its surface. More specifically, the polymer had the structural unit represented by the formula (1) in which R₁ represented a methyl group, and R₂ was represented by the following formula (31), formula (32), formula (33), or formula (34):

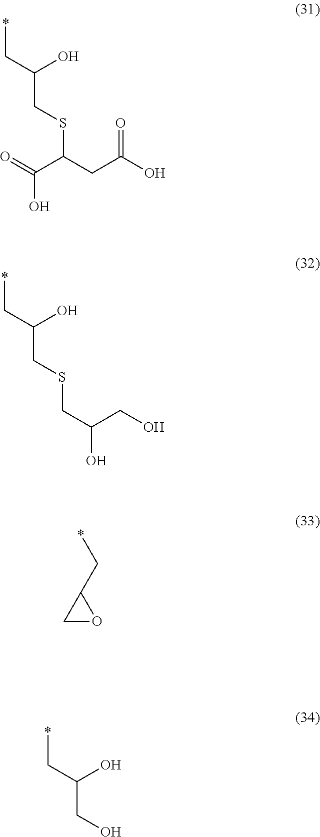

where * represents a bonding position with the structure represented by the formula (1).

TABLE 1

| | Synthesis conditions | | | | | | |
|---|---|---|---|---|---|---|---|
| | Step 1 | | | | Step 2 | Physical properties | |
| | Amount of | Amount | Amount | Stirring | Amount | Volume average | |
| | styrene | of DVB | of V-50 | speed | of GMA | particle diameter | Refractive |
| | (g) | (g) | (g) | (rpm) | (g) | (nm) | index |
| Particle 1 | 85.89 | 1.56 | 3.72 | 200 | 0.39 | 420 | 1.55 |
| Particle 2 | 100.45 | 1.82 | 4.35 | 200 | 0.39 | 500 | 1.55 |
| Particle 3 | 42.81 | 0.78 | 1.86 | 200 | 0.39 | 350 | 1.55 |
| Particle 4 | 110.50 | 2.00 | 4.79 | 200 | 0.39 | 520 | 1.55 |
| Particle 5 | 46.64 | 0.85 | 2.02 | 140 | 0.39 | 300 | 1.55 |
| Particle 6 | 21.53 | 0.39 | 0.93 | 140 | 1.54 | 250 | 1.49 |
| Particle 7 | 36.38 | 0.66 | 1.57 | 140 | 1.54 | 300 | 1.49 |
| Particle 8 | 15.10 | 0.27 | 0.65 | 100 | 1.54 | 200 | 1.49 |
| Particle 9 | 40.02 | 0.72 | 1.73 | 140 | 0.77 | 300 | 1.52 |

| | | | |
|---|---|---|---|
| Particle 10 | “IMMUTEX P0307” manufactured by JSR Life Sciences, LLC | 430 | 1.65 |
| Particle 11 | “IMMUTEX P0113” manufactured by JSR Life Sciences, LLC | 220 | 1.65 |

### (Production of Large Affinity Particle 1)

300 Microliters (3 mg in terms of particle solid content) of the dispersion of the particle 1 diluted with ion-exchanged water so as to have a solid content concentration of 1.0 mass/vol % was aliquoted into a 1.5 mL microtube. 90 Microliters of a 5.0 mass % aqueous solution of 1-(3-dimethylaminopropyl)-3-ethylcarbodiimide hydrochloride and 90 μL of a 5.0 mass % aqueous solution of N-hydroxysulfosuccinimide sodium salt were added thereto. The mixture was stirred at room temperature for 30 minutes to provide a dispersion of a particle having an activated carboxy group (activated particle dispersion).

After centrifugal washing, 270 μL of phosphate-buffered saline (hereinafter referred to as “PBS”) having a pH of 5.5 was added thereto, and the particle having an activated carboxy group was dispersed with an ultrasonic wave.

30 Microliters (0.15 mg in terms of antibody amount) of a 5.0 mg/mL dispersion of a mouse monoclonal anti-ferritin antibody (isoelectric point: 7.1) was added as a ligand thereto, and the mixture was stirred at room temperature for 3 hours so that the particle was sensitized with the antibody. After that, 720 μL of a 1 mol/L tris(hydroxymethyl)aminomethane (Tris) solution having a pH of 8.0 was added as a reaction terminator thereto, and the mixture was allowed to stand still overnight under an environment at 4° C. to provide an antibody-sensitized test particle. After the centrifugal washing of the test particle, 500 μL of PBS was added thereto. Thus, a large affinity particle 1 was obtained.

### (Production of Large Affinity Particle 2 to Small Affinity Particle 15)

A large affinity particle 2 to a small affinity particle 15 were each produced by the same experimental operation as that in the production of the large affinity particle 1 except that, in the method of producing the large affinity particle 1, the kind of the particle and the addition amount of the antibody dispersion were changed as shown in Table 2. The addition amount of the antibody with respect to 1.0 mg of the pre-sensitization particle of each of the obtained large affinity particle 1 to small affinity particle 15, and the zeta potential thereof are collectively shown in Table 2.

TABLE 2

| | Preparation conditions | | Physical properties | |
|---|---|---|---|---|
| | | Addition amount | Amount of antibody with | |
| | | of antibody | respect to 1 mg of pre- | Zeta |
| | Particle | dispersion | sensitization particle | potential |
| | No. | (μL) | (μg/mg) | (mV) |
| Large affinity particle 1 | 1 | 30.0 | 50 | −25 |
| Large affinity particle 2 | 2 | 30.0 | 50 | −21 |
| Large affinity particle 3 | 3 | 30.0 | 50 | −26 |
| Large affinity particle 4 | 4 | 30.0 | 50 | −20 |
| Large affinity particle 5 | 5 | 30.0 | 50 | −26 |
| Large affinity particle 6 | 1 | 40.0 | 66 | −22 |
| Large affinity particle 7 | 10 | 30.0 | 31 | −42 |
| Small affinity particle 8 | 6 | 4.8 | 10 | −35 |
| Small affinity particle 9 | 7 | 4.8 | 10 | −36 |
| Small affinity particle 10 | 8 | 4.8 | 10 | −34 |
| Small affinity particle 11 | 6 | 1.4 | 3 | −37 |
| Small affinity particle 12 | 6 | 7.7 | 16 | −33 |
| Small affinity particle 13 | 9 | 4.8 | 10 | −29 |
| Small affinity particle 14 | 8 | 7.7 | 16 | −33 |
| Small affinity particle 15 | 11 | 4.8 | 6 | −45 |

### (Preparation of Second Reagent 1)

A second reagent 1 was prepared by using the produced large affinity particle 1 and small affinity particle 8. Specifically, the large affinity particle 1 and the small affinity particle 8 were each centrifuged, and re-dispersed in 500 μL of a buffer (HEPES buffer) in which 10 mM HEPES, 0.01 mass % polyoxyethylene nonylphenyl ether (Triton X-100: Kishida Chemical Co., Ltd.), and 5.0 mass % sucrose (viscosity modifier) were dissolved in ion-exchanged water. After that, the contents were mixed and diluted with the HEPES buffer so that the concentrations of the large affinity particle 1 and the small affinity particle 8 were 0.075 mass/vol % and 0.078 mass/vol %, respectively, and the total concentration thereof was 0.153 mass/vol % to provide the second reagent 1.

### (Preparation of Second Reagent 2 to Second Reagent 14)

A second reagent 2 to a second reagent 14 were each prepared by the same experimental operation as that in the preparation of the second reagent 1 except that, in the preparation of the second reagent 1, the kinds of the affinity particles and the concentrations of the affinity particles were changed as shown in Table 3. The physical properties of the obtained second reagent 1 to second reagent 14 are collectively shown in Tables 3-1 and 3-2.

TABLE 3-1

| | | | | Amount of |
|---|---|---|---|---|
| | | | | antibody with |
| | | | Ratio of mass of | respect to |
| | | | large particle to | particle in |
| | Kinds and concentrations of affinity | | mass of small | second reagent |
| | particles | | particle | (μg/mL) |
| Second reagent 1 | Large affinity particle 1 | 0.075 mass/vol % | 0.96 | 29.6 |
| | Small affinity particle 8 | 0.078 mass/vol % | | |
| Second reagent 2 | Large affinity particle 1 | 0.075 mass/vol % | 1.60 | 34.6 |
| | Small affinity particle 8 | 0.047 mass/vol % | | |
| Second reagent 3 | Large affinity particle 2 | 0.060 mass/vol % | 0.86 | 28.5 |
| | Small affinity particle 9 | 0.070 mass/vol % | | |
| Second reagent 4 | Large affinity particle 3 | 0.100 mass/vol % | 1.25 | 32.2 |
| | Small affinity particle 10 | 0.080 mass/vol % | | |
| Second reagent 5 | Large affinity particle 4 | 0.052 mass/vol % | 0.80 | 27.8 |
| | Small affinity particle 9 | 0.065 mass/vol % | | |
| Second reagent 6 | Large affinity particle 5 | 0.120 mass/vol % | 1.20 | 31.8 |
| | Small affinity particle 10 | 0.100 mass/vol % | | |
| Second reagent 7 | Large affinity particle 1 | 0.075 mass/vol % | 2.42 | 38.3 |
| | Small affinity particle 8 | 0.031 mass/vol % | | |
| Second reagent 8 | Large affinity particle 1 | 0.055 mass/vol % | 0.46 | 22.6 |
| | Small affinity particle 8 | 0.120 mass/vol % | | |
| Second reagent 9 | Large affinity particle 6 | 0.075 mass/vol % | 0.96 | 33.9 |
| | Small affinity particle 11 | 0.078 mass/vol % | | |
| Second reagent 10 | Large affinity particle 1 | 0.075 mass/vol % | 0.96 | 32.7 |
| | Small affinity particle 12 | 0.078 mass/vol % | | |
| Second reagent 11 | Large affinity particle 6 | 0.075 mass/vol % | 1.50 | 34.0 |
| | Small affinity particle 13 | 0.050 mass/vol % | | |
| Second reagent 12 | Large affinity particle 2 | 0.060 mass/vol % | 0.35 | 24.9 |
| | Small affinity particle 14 | 0.170 mass/vol % | | |
| Second reagent 13 | Large affinity particle 7 | 0.080 mass/vol % | 0.57 | 15.1 |
| | Small affinity particle 15 | 0.140 mass/vol % | | |
| Second reagent 14 | Large affinity particle 7 | 0.080 mass/vol % | 1.60 | 21.4 |
| | Small affinity particle 15 | 0.050 mass/vol % | | |

TABLE 3-2

| | Ratio between amount of | | | |
|---|---|---|---|---|
| | antibody with respect to | | | |
| | particle concentration of large | Volume | Difference in | Ratio of refractive |
| | particle and amount of antibody | average | volume average | index of large |
| | with respect to particle | particle | particle diameter | particle to refractive |
| | concentration of small particle | diameter | between two | index of small |
| | in second reagent | (nm) | particles (nm) | particle |
| Second reagent 1 | 5.0 | 333 | 170 | 0.96 |
| Second reagent 2 | 5.0 | 355 | 170 | 0.96 |
| Second reagent 3 | 5.0 | 392 | 200 | 0.96 |
| Second reagent 4 | 5.0 | 283 | 150 | 0.96 |
| Second reagent 5 | 5.0 | 398 | 220 | 0.96 |
| Second reagent 6 | 5.0 | 255 | 100 | 0.96 |
| Second reagent 7 | 5.0 | 370 | 170 | 0.96 |
| Second reagent 8 | 5.0 | 303 | 170 | 0.96 |
| Second reagent 9 | 22.0 | 333 | 170 | 0.96 |
| Second reagent 10 | 3.1 | 333 | 170 | 0.96 |
| Second reagent 11 | 5.0 | 372 | 120 | 0.98 |
| Second reagent 12 | 3.1 | 278 | 300 | 0.96 |
| Second reagent 13 | 5.2 | 296 | 210 | 1.00 |
| Second reagent 14 | 5.2 | 349 | 210 | 1.00 |

### (Preparation of First Reagent)

A first reagent was a mixture in which HEPES, Triton X-100, and NaCl were mixed by using ion-exchanged water to concentrations of 50 mM HEPES, 0.10 mass % Triton X-100, and 0.90 mass % NaCl, respectively.

(Magnitude of Amount of Change in Absorbance with Respect to Concentration of Each of Low-Concentration Standard Solution and High-Concentration Standard Solution)

A spectrophotometer BIOSPECTROMETER (Eppendorf AG) was used as a measuring device, and a measurement wavelength was set to 572 nm. A mixed solution was prepared by mixing 15 μL of a specimen and 60 μL of the first reagent 1, and was kept warm for 290 seconds under the condition of 37° C. Next, 30 μL of the second reagent 1 was mixed into the above-mentioned mixed solution, and the mixture was stirred, followed by the measurement of its absorbance 42 seconds after the stirring. Further, the mixed solution was allowed to stand still at 37° C. for 253 seconds, and then its absorbance was measured again. The difference of the absorbance from the absorbance 42 seconds after the stirring was defined as the amount of change in absorbance.

Standard solutions having ferritin concentrations of 0 ng/ml, 10 ng/ml, 90 ng/mL, 100 ng/mL, 1,000 ng/mL, 1,100 ng/mL, 1,400 ng/mL, and 1,500 ng/mL were prepared. The change in absorbance of each of those standard solutions was measured, and the amount of change in absorbance ΔOD was calculated.

When the amount of change in absorbance of a standard solution A containing A ng/mL of ferritin was represented by ΔODA, the amount of change in absorbance of a standard solution B containing B ng/ml of ferritin was represented by ΔODB (B>A), and a relationship of symbols was represented by (ΔODB−ΔODA)/(B−A)=X [A−B], the magnitude of the amount of change in absorbance with respect to the concentration of the low-concentration standard solution was calculated as X [0-10] and X [90-100], and the magnitude of the amount of change in absorbance with respect to the concentration of the high-concentration standard solution was calculated as X [1,000-1,100] and X [1,400-1,500]. Those experiments were performed for each second reagent used in each Example and Comparative Example. In addition, the absorbance measured 42 seconds after mixing the second reagent and stirring the mixture in the measurement of the standard solution having a ferritin concentration of 0 ng/mL was defined as an initial absorbance at 0 ng/mL. Those results are collectively shown in Table 4.

TABLE 4

| | | | Difference in amount of | | Difference in amount of change | |
|---|---|---|---|---|---|---|
| | | | change in absorbance with | | in absorbance with respect to | |
| | | | respect to concentration of | | concentration of high- | |
| | | Initial | low-concentration standard | | concentration standard solution | |
| | | absorbance | solution | | X [1,000- | X [1,400- |
| | | at 0 ng/ml | X [0-10] | X [90-100] | 1,100] | 1,500] |
| Example 1 | Second | 1.55 | 0.00152 | 0.00082 | 0.00008 | 0.00007 |
| | reagent 1 | | | | | |
| Example 2 | Second | 1.48 | 0.00157 | 0.00079 | 0.00007 | 0.00005 |
| | reagent 2 | | | | | |
| Example 3 | Second | 1.80 | 0.00152 | 0.00083 | 0.00006 | 0.00003 |
| | reagent 3 | | | | | |
| Example 4 | Second | 1.87 | 0.00118 | 0.00071 | 0.00009 | 0.00004 |
| | reagent 4 | | | | | |
| Example 5 | Second | 1.94 | 0.00217 | 0.00080 | 0.00004 | 0.00002 |
| | reagent 5 | | | | | |
| Example 6 | Second | 1.75 | 0.00104 | 0.00066 | 0.00010 | 0.00006 |
| | reagent 6 | | | | | |
| Example 7 | Second | 1.70 | 0.00162 | 0.00075 | 0.00004 | 0.00003 |
| | reagent 7 | | | | | |
| Example 8 | Second | 1.59 | 0.00116 | 0.00055 | 0.00011 | 0.00007 |
| | reagent 8 | | | | | |
| Example 9 | Second | 1.55 | 0.00229 | 0.00124 | 0.00003 | 0.00003 |
| | reagent 9 | | | | | |
| Example 10 | Second | 1.55 | 0.00118 | 0.00079 | 0.00004 | 0.00002 |
| | reagent 10 | | | | | |
| Example 11 | Second | 1.64 | 0.00131 | 0.00092 | 0.00003 | 0.00001 |
| | reagent 11 | | | | | |
| Comparative | Second | 2.17 | 0.00115 | 0.00040 | 0.00017 | 0.00011 |
| Example 1 | reagent 12 | | | | | |
| Comparative | Second | 1.90 | 0.00078 | 0.00044 | 0.00010 | 0.00008 |
| Example 2 | reagent 13 | | | | | |
| Comparative | Second | 1.44 | 0.00093 | 0.00050 | Less than 0 | Less than 0 |
| Example 3 | reagent 14 | | | | | |

### (Evaluation Method for Average Value of Amounts of Change in Absorbance ΔOD of Low-Concentration Ferritin Specimen)

A discrete clinical chemistry automatic analyzer TBA-120FR (Canon Medical Systems Corporation) was used as a measuring device. The optical path length of the device is 5 mm, but an absorbance converted to that in the case where the optical path length of 1 cm is obtained as a measured value. PBS (not containing ferritin) and a specimen C containing 9.8 ng/ml of ferritin were each used as a specimen. Measurement was performed under the following assay parameter conditions: specimen: 15 μL; first reagent 1:60 μL; second reagent: 30 μL; and measurement wavelength: 572 nm. The value of a difference OD(33)−OD(19) between an absorbance OD(19) at photometric point 19 (about 40 seconds after mixing the second reagent) and an absorbance OD(33) at photometric point 33 (about 300 seconds after mixing the second reagent) of the obtained reaction curve was calculated as the amount of change in absorbance ΔOD. The measurement was performed 20 times each, and the average value of the amounts of change in absorbance ΔOD of PBS and the average value of the amounts of change in absorbance ΔOD of the specimen C were calculated. A value obtained by subtracting the average value of the amounts of change in absorbance ΔOD in the case of using physiological saline from the average value of the amounts of change in absorbance ΔOD in the case of using the specimen C was defined as the average value of the amounts of change in absorbance ΔOD for 9.8 ng/mL.

The average value of the amounts of change in absorbance ΔOD for 9.8 ng/ml obtained in this manner was evaluated in accordance with the following criteria. The results are summarized in Table 5 below.
A: The ΔOD average was 0.00160 or more.
B: The ΔOD average was 0.00130 or more and less than 0.00160.
C: The ΔOD average was 0.00100 or more and less than 0.00130.
D: The ΔOD average was 0.00080 or more and less than 0.00100.
E: The ΔOD average was less than 0.00080.

### (Evaluation Method for Measurement Reproducibility of Low-Concentration Ferritin Specimen)

A discrete clinical chemistry automatic analyzer TBA-120FR (Canon Medical Systems Corporation) was used as a measuring device. A specimen C containing 9.8 ng/mL of ferritin was subjected to measurement under the following assay parameter conditions: specimen: 15 μL; first reagent 1:60 μL; second reagent: 30 μL; and measurement wavelength: 572 nm. The value of a difference OD(33)−OD(19) between an absorbance OD(19) at photometric point 19 and an absorbance OD(33) at photometric point 33 of the obtained reaction curve was calculated as the amount of change in absorbance ΔOD. The measurement was performed 20 times, and the standard deviation (hereinafter referred to as “SD”) of the amounts of change in absorbance ΔOD was calculated.

The SD of the amounts of change in absorbance ΔOD for the specimen C having a concentration of 9.8 ng/mL obtained in this manner was evaluated in accordance with the following criteria. The results are summarized in Table 5 below.
A: The SD was less than 3.0.
B: The SD was 3.0 or more and less than 4.0.
C: The SD was 4.0 or more and less than 5.0.
D: The SD was 5.0 or more and less than 7.0.
E: The SD was 7.0 or more and less than 10.0.
F: The SD was 10.0 or more.

### (Creation of Calibration Curve)

A discrete clinical chemistry automatic analyzer TBA-120FR (Canon Medical Systems Corporation) was used as a measuring device. First, standard solutions having ferritin concentrations of 0 ng/mL, 100 ng/ml, 250 ng/ml, 500 ng/ml, 1,000 ng/ml, 1,500 ng/mL, and 2,000 ng/ml were prepared. Those standard solutions each serving as a specimen were measured under the following assay parameter conditions: specimen: 15 μL; first reagent 1:60 μL; second reagent: 30 μL; and measurement wavelength: 572 nm. The value of a difference OD(33)−OD(19) between an absorbance OD(19) at photometric point 19 and an absorbance OD(33) at photometric point 33 in the obtained reaction curve was calculated as the amount of change in absorbance ΔOD. A calibration curve showing a relationship between each ferritin concentration and ΔOD was created. Those experiments were performed for each second reagent used in each Example.

### (Evaluation Method for Measurement Reproducibility of High-Concentration Ferritin Specimen)

A discrete clinical chemistry automatic analyzer TBA-120FR (Canon Medical Systems Corporation) was used as a measuring device. A specimen D containing 1,515.0 ng/ml of ferritin was subjected to measurement under the following assay parameter conditions: specimen: 15 μL; first reagent 1:60 μL; second reagent: 30 μL; and measurement wavelength: 572 nm. The value of a difference OD(33)−OD(19) between an absorbance OD(19) at photometric point 19 and an absorbance OD(33) at photometric point 33 of the obtained reaction curve was calculated as the amount of change in absorbance ΔOD. The measurement was performed 20 times, a ferritin concentration was quantified by using the calibration curve, which had been created in advance, and the SD of the obtained ferritin-converted concentrations was calculated.

The SD of the ferritin-converted concentrations for the specimen D having a concentration of 1,515.0 ng/mL obtained in this manner was evaluated in accordance with the following criteria. The results are summarized in Table 5 below.
A: The SD was less than 15.0.
B: The SD was 15.0 or more and less than 18.0.
C: The SD was 18.0 or more and less than 21.0.
D: The SD was 21.0 or more and less than 24.0.
E: The SD was 24.0 or more and less than 27.0.
F: The SD was 27.0 or more.

TABLE 5

| | | Low-concentration specimen evaluation | | High-concentration |
|---|---|---|---|---|
| | | | SD of amounts of | specimen evaluation |
| | | Average value of | change in | SD of ferritin- |
| | | differences in | absorbance ΔOD | converted |
| | | amount of change | for specimen C | concentrations for |
| | | in absorbance | having | specimen D having |
| | | ΔOD for 9.8 | concentration of | concentration of |
| | | ng/mL | 9.8 ng/mL | 1,515.0 ng/mL |
| Example 1 | Second reagent 1 | A | A | A |
| Example 2 | Second reagent 2 | B | A | A |
| Example 3 | Second reagent 3 | A | A | A |
| Example 4 | Second reagent 4 | C | A | A |
| Example 5 | Second reagent 5 | A | C | B |
| Example 6 | Second reagent 6 | C | B | C |
| Example 7 | Second reagent 7 | A | C | C |
| Example 8 | Second reagent 8 | C | C | C |
| Example 9 | Second reagent 9 | A | A | C |
| Example 10 | Second reagent 10 | C | C | B |
| Example 11 | Second reagent 11 | B | B | C |
| Comparative Example 1 | Second reagent 12 | C | D | E |
| Comparative Example 2 | Second reagent 13 | E | E | C |
| Comparative Example 3 | Second reagent 14 | D | D | F |

Those results show that the reagent satisfying the definition of the present disclosure provides an excellent detection limit and enables highly reproducible measurement even in the high-concentration region.

Ferritin has been described as an example of the target substance that may be used in the present disclosure. A protein in which a plurality of subunits are associated such as ferritin has a plurality of ligand recognition sites, and hence the ligand recognition sites tend to remain even when the aggregation reaction of the particles progresses. When the particle concentration in the reaction system is high, multiparticle aggregation in which multiple particles react with one target substance is conceived to be likely to occur. From this viewpoint, the measurement method of the present disclosure may be said to target a substance having a plurality of reaction sites for a ligand that specifically binds to the target substance.

The present disclosure can provide the measurement method that improves a detection limit and enables highly reproducible measurement even at a high concentration with a protein that is hexameric or higher as a target substance by immunoturbidimetry using a particle, and the reagent therefor.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2024-218590, filed Dec. 13, 2024, and Japanese Patent Application No. 2025-185679, filed Nov. 4, 2025, which are hereby incorporated by reference herein in their entirety.

What is claimed is:

1. A measurement method for measuring a target substance in a specimen,
the target substance being a protein forming a complex that is hexameric or higher,
the measurement method comprising:
a first mixing step of mixing the specimen and a first reagent to provide a first mixed solution;
a second mixing step of mixing the first mixed solution and a second reagent, which includes a particle having immobilized thereon a ligand that specifically binds to the target substance, to provide a second mixed solution; and
a measurement step of measuring an absorbance of the second mixed solution,
wherein the absorbance at a time point of 35 seconds or more and 45 seconds or less from the mixing in the second mixing step is 1.40 or more,
wherein, when an amount of change in absorbance of a specimen A containing A ng/mL of the target substance is represented by ΔODA, an amount of change in absorbance of a specimen B containing B ng/ml of the target substance is represented by ΔODB (B>A), and a relationship of symbols is represented by (ΔODB−ΔODA)/(B−A)=X,
an X of 0.00100 or more is present in a range where both A and B are 0 or more and 100 or less, and
an X of 0.00003 or more and 0.00010 or less is present in a range where both A and B are 1,000 or more and 1,500 or less.

2. The measurement method according to claim 1, wherein the specimen is selected from the group consisting of blood, serum, and plasma each including the target substance.

3. The measurement method according to claim 1, wherein the target substance has a molecular weight of 300 kDa or more and 600 kDa or less.

4. The measurement method according to claim 1, wherein the target substance is ferritin.

5. The measurement method according to claim 1, wherein the particle has a volume average particle diameter of 200 nm or more and 500 nm or less.

6. The measurement method according to claim 1, wherein the particle is a particle obtained by mixing two kinds of particles having different volume average particle diameters, and a difference in volume average particle diameter between the two kinds of particles is 250 nm or less.

7. The measurement method according to claim 1, wherein the particle is a particle obtained by mixing two kinds of particles having different volume average particle diameters, and a volume average particle diameter of the particle having a larger volume average particle diameter is 350 nm or more and 500 nm or less.

8. The measurement method according to claim 1, wherein the particle is a particle obtained by mixing two kinds of particles having different volume average particle diameters, and a volume average particle diameter of the particle having a smaller volume average particle diameter is 200 nm or more and 300 nm or less.

9. The measurement method according to claim 1, wherein a concentration of the particle in the second mixed solution is 0.02 mass/vol % or more and 0.10 mass/vol % or less.

10. The measurement method according to claim 1, wherein the particle is a particle obtained by mixing two kinds of particles having different volume average particle diameters, and a concentration of the particle having a larger volume average particle diameter in the second reagent is 0.5 times or more and 2.0 times or less as high as a concentration of the particle having a smaller volume average particle diameter.

11. The measurement method according to claim 1, wherein an amount of the ligand with respect to 1.0 mg of a pre-sensitization particle excluding the ligand from the particle in the second reagent is 10.0 μg/mg or more and 40.0 μg/mg or less.

12. The measurement method according to claim 11, wherein the particle is a particle obtained by mixing two kinds of particles having different volume average particle diameters, and an amount of the ligand with respect to 1.0 mg of the pre-sensitization particle of the particle having a larger volume average particle diameter is 4.0 times or more and 20.0 times or less as large as an amount of the ligand with respect to 1.0 mg of the pre-sensitization particle of the particle having a smaller volume average particle diameter.

13. The measurement method according to claim 1, wherein the ligand is an antibody having an isoelectric point of 5.0 or more and 8.0 or less.

14. The measurement method according to claim 1, wherein the particle is a particle obtained by mixing two kinds of particles having different volume average particle diameters, and a ratio of a refractive index of the particle having a larger volume average particle diameter to a refractive index of the particle having a smaller volume average particle diameter is 0.87 or more and 0.97 or less.

15. The measurement method according to claim 1, wherein the particle is a particle obtained by mixing two kinds of particles having different volume average particle diameters, and a zeta potential of the particle having a larger volume average particle diameter is −35.0 mV or more and −15.0 mV or less.

16. The measurement method according to claim 1, wherein the particle is a particle obtained by mixing two kinds of particles having different volume average particle diameters, and a zeta potential of the particle having a smaller volume average particle diameter is −40.0 mV or more and −25.0 mV or less.

17. The measurement method according to claim 1, wherein the particle contains a polymer having a structure represented by the following formula (1): 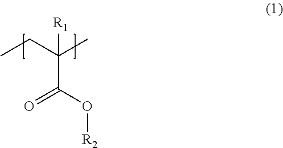
where R₁ represents a methyl group or a hydrogen atom, and R₂ represents a group having any one of an epoxy group, a hydroxy group, or a carboxy group.

18. The measurement method according to claim 17, wherein the formula (1) represents a structure represented by the following formula (1-A): 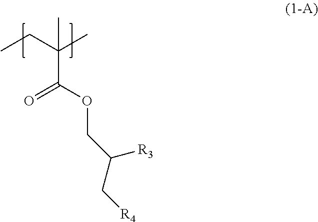
where at least one of R₃ or R₄ represents a hydroxy group, and another thereof represents a hydroxy group or a group represented by the following formula (1-B): 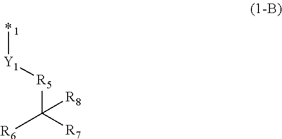
where R₅ represents a single bond or a methylene group, R₆, R₇, and R₈ are each selected from a hydrogen atom, a methyl group, a hydroxy group, a carboxy group, a hydroxymethyl group, and a carboxymethyl group, and at least one of R₆, R₇, or R₈ includes a hydroxy group or a carboxy group, Y₁ represents a sulfur atom or an imino group, and *₁ represents a bonding position with the structure represented by the formula (1-A).

19. The measurement method according to claim 1, wherein a measurement wavelength of the absorbance includes 500 nm or more and 750 nm or less.

20. The measurement method according to claim 1, wherein the measurement step includes calculating an amount of change in absorbance ΔOD from a difference between the absorbance within a range of 15 seconds or more and 45 seconds or less from the mixing in the second mixing step, and the absorbance within a range of 250 seconds or more and 300 seconds or less from the mixing in the second mixing step.

21. The measurement method according to claim 1, wherein a concentration of the specimen in the second mixed solution is 7.0 vol % or more and 30.0 vol % or less.

22. The measurement method according to claim 1, wherein the first reagent and the second reagent each include at least one of a sugar or a sugar alcohol.

23. The measurement method according to claim 1, wherein the first reagent and the second reagent each include one or more surfactants selected from the group consisting of a sorbitan fatty acid ester, a polyoxyethylene alkyl ether, and a polyoxyethylene phenyl ether.

24. The measurement method according to claim 1, wherein the target substance is a substance having a plurality of reaction sites for the ligand that specifically binds to the target substance.

25. A reagent for measuring a target substance in a specimen for use in the measurement method according to claim 1,
the target substance being a protein forming a complex that is hexameric or higher,
the reagent comprising a first reagent including a buffering agent, and a second reagent including a particle having immobilized thereon a ligand that specifically binds to the target substance,
wherein, when a first mixing step of obtaining a first mixed solution, in which the specimen and the first reagent are mixed at a volume ratio “specimen:first reagent” of 15:60, and a second mixing step of obtaining a second mixed solution, in which the first mixed solution and the second reagent are mixed at a volume ratio “first mixed solution:second reagent” of 75:30, are performed,
an absorbance of the second mixed solution at a time point of 35 seconds or more and 45 seconds or less from the mixing in the second mixing step when using the specimen containing 0 ng/mL of the target substance is 1.40 or more, and
wherein, when an amount of change in absorbance of the second mixed solution from 40 seconds to 300 seconds from the mixing in the second mixing step when using the specimen containing 0 ng/mL of the target substance is represented by ΔOD(0), an amount of change in absorbance of the second mixed solution from 40 seconds to 300 seconds from the mixing in the second mixing step when using the specimen containing 10 ng/ml of the target substance is represented by ΔOD(10), an amount of change in absorbance of the second mixed solution from 40 seconds to 300 seconds from the mixing in the second mixing step when using the specimen containing 90 ng/ml of the target substance is represented by ΔOD(90), an amount of change in absorbance of the second mixed solution from 40 seconds to 300 seconds from the mixing in the second mixing step when using the specimen containing 100 ng/mL of the target substance is represented by ΔOD(100), an amount of change in absorbance of the second mixed solution from 40 seconds to 300 seconds from the mixing in the second mixing step when using the specimen containing 1,000 ng/ml of the target substance is represented by ΔOD(1,000), an amount of change in absorbance of the second mixed solution from 40 seconds to 300 seconds from the mixing in the second mixing step when using the specimen containing 1,100 ng/mL of the target substance is represented by ΔOD(1,100), an amount of change in absorbance of the second mixed solution from 40 seconds to 300 seconds from the mixing in the second mixing step when using the specimen containing 1,400 ng/ml of the target substance is represented by ΔOD(1,400), and an amount of change in absorbance of the second mixed solution from 40 seconds to 300 seconds from the mixing in the second mixing step when using the specimen containing 1,500 ng/ml of the target substance is represented by ΔOD(1,500),
a value of (ΔOD(10)−ΔOD(0))/(10−0) or (ΔOD(100)−ΔOD(90))/(100−90) is 0.00100 or more, and
a value of (ΔOD(1,100)−ΔOD(1,000))/(1,100−1,000) or (ΔOD(1,500)−ΔOD(1,400))/(1,500−1,400) is 0.00003 or more and 0.00010 or less.
